(19) United States
(12) Patent Application Publication
(10) Pub. No.: US 2025/0378961 A1
(43) Pub. Date: Dec. 11, 2025

(54) SYSTEM AND METHOD FOR AUTOMATIC AND/OR DATA-DRIVEN PERITUMORAL INFILTRATION RISK STRATIFICATION

(71) Applicant: Case Western Reserve University, Cleveland, OH (US)

(72) Inventors: Dan Ma, Solon, OH (US); Walter Zhao, Cleveland Heights, OH (US)

(21) Appl. No.: 19/192,686

(22) Filed: Apr. 29, 2025

Related U.S. Application Data
(60) Provisional application No. 63/642,607, filed on May 3, 2024.

(51) Int. Cl. G16H 50/30; G06T 7/00; G06T 7/11; G06V 10/25; G16H 10/60; G16H 15/00; G16H 30/20; G16H 30/40

(52) U.S. Cl. CPC G16H 50/30; G06T 7/0012; G06T 7/11; G06V 10/25; G16H 10/60; G16H 15/00; G16H 30/20; G16H 30/40; G06T 2207/10088; G06T 2207/30096

(57) ABSTRACT

The present disclosure provides systems and methods for automatic peritumoral infiltration risk stratification. A method includes obtaining medical image data of a tumor, segmenting the medical image data into a tumor core region and a peritumoral zone region, and projecting voxel data from the tumor core region and the peritumoral zone region into a feature space. The method can also include identifying a characteristic image data of the tumor core region as a centroid of the tumor core region voxels within the feature space, comparing voxel feature data of the peritumoral zone region to the tumor core region centroid to rank voxels of the peritumoral zone region based on similarity to the tumor core region data, and using a modified triplet loss function to grow two distinct regions of interest within the peritumoral zone region.

### STATEMENT REGARDING FEDERALLY SPONSORED RESEARCH

This invention was made with government support under CA269604 and NS109439 awarded by the National Institutes of Health. The government has certain rights in the invention.

### BACKGROUND

Medical imaging plays a crucial role in the diagnosis, treatment planning, and monitoring of various diseases, particularly in oncology. Magnetic resonance imaging (MRI) has emerged as a powerful tool for visualizing soft tissues and providing detailed anatomical information. In the field of neuro-oncology, MRI is widely used to assess brain tumors and their surrounding environment.

The tumor microenvironment (TME) encompasses the area immediately surrounding a tumor and includes various components such as blood vessels, immune cells, and stromal cells. Understanding the TME is becoming increasingly recognized as an important factor in tumor growth, progression, and response to treatment. Within the TME, the peritumoral zone (PZ) represents a transitional area between the tumor core and healthy tissue.

Characterizing the PZ can provide valuable insights into tumor behavior and potential infiltration into surrounding tissues. However, accurately delineating and analyzing the PZ remains challenging due to its heterogeneous nature and subtle differences from both tumor and healthy tissue. Traditional imaging techniques may not fully capture the complexities of the PZ, leading to potential underestimation or overestimation of tumor extent.

### SUMMARY

In one, non-limiting aspect of the present disclosure, a method for automatic peritumoral infiltration risk stratification is provided that includes obtaining medical image data of a tumor, segmenting the medical image data into a tumor core region and a peritumoral zone region, and projecting voxel data from the tumor core region and the peritumoral zone region into a feature space. The method also includes identifying a characteristic image data of the tumor core region as a centroid of the tumor core region voxels within the feature space and comparing voxel feature data of the peritumoral zone region to the tumor core region centroid to rank voxels of the peritumoral zone region based on similarity to the tumor core region data. The method further includes using a modified triplet loss function to grow two distinct regions of interest within the peritumoral zone region, and generating a report of peritumoral infiltration risk stratification using results of the modified triplet loss function to grow two distinct regions of interest within the peritumoral zone region.

In another non-limiting aspect of the present disclosure, a system is provided for automatic peritumoral infiltration risk stratification. The system includes a processor and a memory storing instructions that, when executed by the processor, cause the system to obtain medical image data of a tumor, segment the medical image data into a tumor core region and a peritumoral zone region, and project voxel data from the tumor core region and the peritumoral zone region into a feature space. The system is further caused to identify a characteristic image data of the tumor core region as a centroid of the tumor core region voxels within the feature space, compare each peritumoral zone region voxel's feature data to the tumor core region centroid to rank the peritumoral zone region voxels based on their similarity to the tumor core region data, and use a modified triplet loss function to grow two distinct regions of interest within the peritumoral zone region. The system can generate a report using the two distinct regions of interest within the peritumoral zone region, wherein the report includes a high-risk region segmented by region growing peritumoral zone region areas with high tumor core region similarity, and a low-risk region generated using peritumoral zone region voxels with low tumor core region similarity.

In yet another non-limiting aspect of the present disclosure, a non-transitory computer-readable medium is provided storing instructions that, when executed by a processor, cause the processor to perform a method for automatic peritumoral infiltration risk stratification. The method includes obtaining medical image data of a tumor, segmenting the medical image data into a tumor core region and a peritumoral zone region, and projecting voxel data from the tumor core region and the peritumoral zone region into a feature space. The method further includes identifying a characteristic image data of the tumor core region as a centroid of the tumor core region voxels within the feature space, comparing each peritumoral zone region voxel's feature data to the tumor core region centroid to rank the peritumoral zone region voxels based on their similarity to the tumor core region data, and using a modified triplet loss function to grow two distinct regions of interest within the peritumoral zone region, wherein the two distinct regions include at least a high-risk region segmented by region growing peritumoral zone region areas with high tumor core region similarity, and a low-risk region generated using peritumoral zone region voxels with low tumor core region similarity.

The above are non-limiting examples. Other features and aspects are described herein.

### BRIEF DESCRIPTION OF THE DRAWINGS

FIG. 1 is a workflow diagram for illustrated with respect to the non-limiting example of peritumoral infiltration risk stratification, according to aspects of the present disclosure.

FIG. 2 is a set of plots showing a triplet loss-based sequential segmentation method, in accordance with the present disclosure.

FIG. 3A is a set of non-limiting example images acquired to study peritumoral infiltration risk analysis, in accordance with the present disclosure.

FIG. 3B is a flowchart setting forth some non-limiting example steps in accordance with the present disclosure.

FIG. 4A set of images showing shape and location of tumors. TripleSeq-generated priors show high shape and location similarity to radiologist manual segmentation: high-risk priors were located adjacent to ET and in T1w-Gd and FLAIR hyperintense region while low-risk priors were located far from ET with low T1w-Gd signal.

FIG. 4B is a set of graphs providing a comparison of radiologist and automated risk assessment results for tumor regions, according to the present disclosure.

FIG. 5A is a table of validation versus discovery data for two cohorts.

FIG. 5B is a table performance evaluation across imaging acquisitions.

FIG. 5C is a set of images showing tumor segmentation and infiltration prediction in accordance with the present disclosure.

FIG. 5D is an image showing brain tumor pathology, according to an aspect of the present disclosure.

FIG. 6 is a block diagram of an MRI system, configured in accordance with the present disclosure.

FIG. 7A is a block diagram of a system architecture for image processing and automation, according to aspects of the present disclosure.

FIG. 7B is a detailed system architecture with interconnected components, in accordance the present disclosure.

### DETAILED DESCRIPTION

The tumor microenvironment (TME) plays an essential role in tumor growth and progression. Defined as the “ecosystem” surrounding a tumor, the TME includes immune cells, stroma, and vasculature. While not tumor tissue itself, the TME provides a hospitable environment for tumorigenesis to occur. While most cancer research has focused on identifying key differences between tumor and non-tumor areas, recent TME work has increasingly recognized the importance of a third region called the peritumoral zone (PZ). The PZ consists of heterogeneous tissue surrounding the tumor and represents the interface between tumor and non-tumor. As a result, the PZ is known to have unique physical and immune signatures unseen in either fully malignant or benign tissue. It is hypothesized that in some tumors, the PZ serves as a “soil bed” where infiltrating tumor cells proliferate and spread to surrounding healthy tissue. Identification of PZ infiltration thus has significant clinical importance for improved tumor prognosis, treatment, and management.

Medical imaging offers the potential to non-invasively identify areas of PZ infiltration, which when combined with artificial intelligence (AI) methods could be used to evaluate tumor invasion and spread (prognosis), expand surgical resection margins (treatment), and evaluate the effectiveness of radiotherapy (management). However, due to the difficulty in identifying ground truth (pathologically confirmed) areas of PZ infiltration, existing tumor infiltration prediction models often employ regions of interest (ROIs) of high- and low-risk infiltrative potential. These “infiltration risk (IR) priors” are manually segmented by expert physicians and are selected using domain knowledge metrics such as distance from the tumor core (TC) margin. Because they are determined following subjective PZ evaluation, IR priors can be considered as risk assessments in contrast to pathologically confirmed areas of PZ infiltration. While promising results have been achieved, manual segmentation possesses inherent disadvantages including the need for expert guidance (e.g., by board-certified physicians), high inter-reader variability, and long segmentation time. These factors make development and clinical translation of tumor infiltration models challenging.

Magnetic Resonance Imaging (MRI) is a medical imaging modality that can create detailed anatomical, functional, and quantitative images of the human body. During an MRI scan, a pulse sequence is played out to generate, spatially encode, and receive tissue-dependent radio-frequency signals. A typical pulse sequence comprises radiofrequency (RF) pulses, gradient waveforms and analog-to-digital converter (ADC) events. The order, timing and properties of these events are controlled by a computer program. The RF pulses and gradient waveforms are transmitted using electrical coils surrounding or positioned near the imaging volume. The generated signal is received using RF coils and digitized using ADC circuitry. Finally, the digital signals are processed using a computer program to reconstruct images that are post-processed and displayed to the operator.

One of the advantages of MRI is that it can provide a large variety of image contrasts for the same underlying anatomical or physiological state. The images can be made sensitive to different contrast mechanisms by simply modifying the pulse sequence type and parameters. Many pulse sequences exist, including gradient echo (GRE), fast low angle shot (FLASH), spin echo (SE), fast spin echo (FSE), echo-planar imaging (EPI), fluid-attenuated inversion recovery (FLAIR), steady state free precession (SSFP), and so forth. These pulse sequences can be further tuned by setting sequence parameters, such as flip angle (FA), repetition time (TR), echo time (TE), inversion time (TI), partial Fourier level, receiver bandwidth, and so forth, that modify contrast levels, such as T1-weighting, T2-weighting, T₂*-weighting, proton density weighting, and so on. Moreover, these sequences can be combined with other preparatory sequences or temporal repetition that generate other contrasts to provide various types of imaging, including magnetization preparation (MP), diffusion weighted imaging (DWI), perfusion weighted imaging (PWI), diffusion tensor imaging (DTI), magnetic resonance fingerprinting (MRF), functional imaging (fMRI), arterial spin labeling (ASL), susceptibility weighted imaging (SWI), and so on. Imaging can also be performed with the presentation of exogenous contrast agents, like gadolinium (Gd)-, iron-, or manganese-based compounds, which affect relaxation times. Thus, there is a large variety of pulse sequences and imaging protocols to choose from.

However, despite the flexibility of MRI as a modality, the above-described factors that make development and clinical translation of tumor infiltration models challenging persist. To address these challenges, the present disclosure provides systems and methods for a triplet loss sequential segmentation for automatic and data-driven peritumoral infiltration risk stratification. These systems and methods, which may be referred to herein as TripleSeq, present a data-driven way to automatically stratify the PZ region into areas of high and low infiltration risk.

The present disclosure provides systems and methods that, while described using a specific tumor type (glioblastoma or GBM) and magnetic resonance imaging (MRI) data, can be employed for peritumor infiltration risk stratification of any tumor type (GBM, hepatocellular carcinoma, etc.) and using any imaging modality of choice (e.g., MRI, computed tomography (CT), positron emission tomography (PET), etc.). That is, regardless of input, the systems and methods provided herein can stratify PZ areas into areas of high and low infiltration risk based on differences in medical image data.

Prior studies exploring the relationship between MRI features and PZ infiltration have identified associations between informative image features and key pathological tumor changes such as increased cellularity or hypervascularization. For example, reduced T2-weighted (T2w) and fluid-attenuated inversion recovery (FLAIR) signal (relative within the PZ) have been shown to predict infiltration and future recurrence, and potentially reflect lower water content and greater tumor cell presence. Other studies have similarly demonstrated elevated T1w-Gd and perfusion MRI metrics in abnormal PZ, indicating enhanced vascular density in infiltrated areas. These findings indicate that infiltrated PZ areas possess MRI characteristics similar to the TC: PZ areas with higher image similarity to TC are more likely to be infiltrated compared to PZ that is more distinct from TC. Importantly, this hypothesis agrees with results from a biopsy-based study of tumor cellularity. Thus, the present disclosure provides systems and methods to automatically identify PZ areas that are representative of both infiltration (similar to TC) and non-infiltration (dissimilar to TC) through quantitative analysis of PZ MRI data.

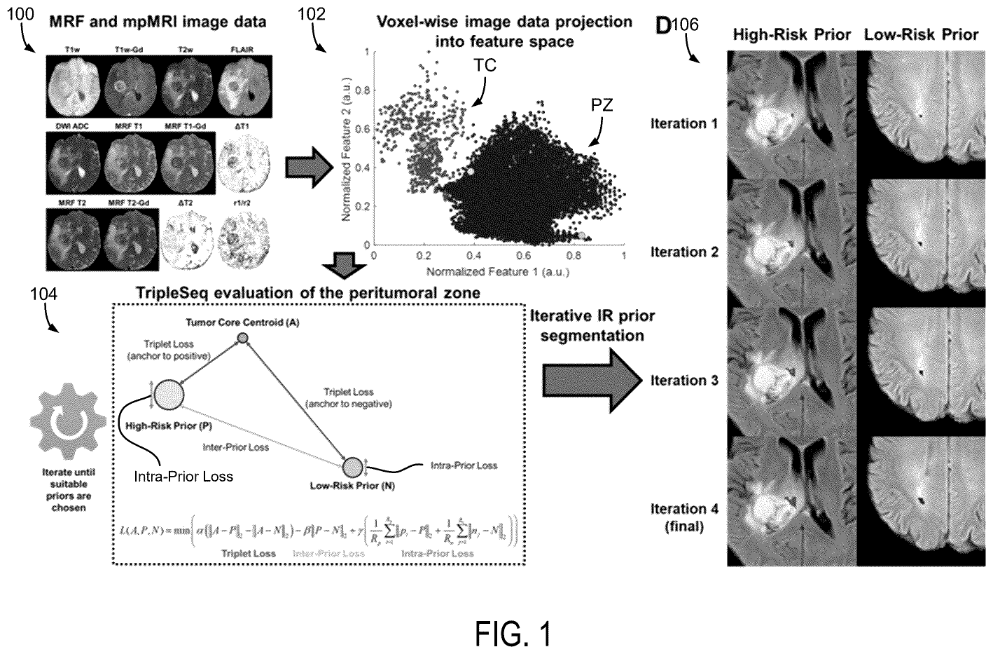

TripleSeq, or triplet loss-based sequential segmentation, employs iterative region growing using modified triplet loss to automatically identify IR priors within the PZ region. Referring to FIG. 1, at block 100, following image acquisition, MRI images are coregistered and segmented to separate tumor into TC (enhancing region on contrast MRI) and PZ (T2w and FLAIR hyperintensity surrounding TC) regions. At block 102, voxel data from TC and PZ are then projected into a high-dimensional feature space, where each data point represents the MRI image data for a given voxel. Next, referring to block 104, the TripleSeq algorithm is used to identify PZ regions with high and low infiltration risk. First, the characteristic MRI image data of TC voxels is obtained as the centroid of TC voxels within the feature space. Next, each PZ voxel's feature data is compared to the TC centroid to rank PZ voxels based on their similarity to TC data. A modified triplet loss function is then used to grow two distinct ROIs within the PZ. A high-risk prior is segmented by region growing PZ areas with high TC similarity, while a low-risk prior is simultaneously generated using PZ voxels with low TC similarity, as illustrated at block 106. The triplet loss is specifically modified to include an inter-prior and an intra-prior loss, which are included to ensure that high- and low-risk ROIs remain distinct from each other (inter-prior loss) while retaining consistent voxel-wise features (intra-prior loss).

More particularly, the systems and methods provided herein for automatic, data-driven PZ infiltration risk (IR) stratification can includes the application of TripleSeq for PZ analysis of GBM tumors using MRI data. In block 100 of FIG. 1, MRI data for a single GBM tumor is coregistered and skull stripped. At block 102, tumor core (TC; red) and peritumoral zone (PZ; blue) image data is then projected voxel-wise into a high-dimensional feature space, where each dimension represents an image feature such as T1w signal intensity. The TC data centroid (cyan) is initialized and used to initialize a high-risk feature vector (yellow) and low-risk feature vector (green). Referring to block 104, TripleSeq employs a modified triplet loss function to perform fully data-driven, automatic region growing of IR priors (high- and low-risk). In addition to standard triplet loss, inter-prior and intra-prior loss terms are added to ensure IR priors remain distinct from each other (inter-prior) while maintaining internal ROI consistency (intra-prior). Referring to block 106, visualization of TripleSeq IR prior segmentation during each region growing iteration. Following the initialization of the high- and low-risk prior ROIs, region growing is performed until automatically terminating once a stopping criterion (inter-prior or intra-prior thresholds) is reached.

Following whole tumor segmentation into TC (defined as the enhancing region on contrast MRI) and PZ (defined as the T2w and FLAIR hyperintense area surrounding the TC) regions, whole tumor (TC and PZ) MRI image data I∈ [symbol] can be projected voxel-wise into a high-dimensional image feature space F∈ [symbol] where m is the number of image features (e.g., T1w intensity) and k is the number of whole tumor (TC and PZ) voxels. The characteristic TC feature vector A∈ [symbol] can then be obtained by calculating the feature space centroid of TC region image voxels, which is used to iteratively search the PZ region for high-risk and low-risk ROIs via modified triplet loss. First, the L2 (Euclidean) distance between each PZ voxel's feature vector and A can be calculated in order to rank PZ voxels based on their similarity to TC data (lower L2 distance means higher similarity). Characteristic high-risk P∈ [symbol] and low-risk N∈ [symbol] feature vectors, representing the feature space centroids of high- and low-risk prior image data, can then be initialized as the feature vectors of the PZ voxels most similar and least similar to TC respectively. With A as anchor (TC), P as positive input (high-risk prior), and N as negative input (low-risk prior), region growing is performed using a modified triplet loss:

$$L ( A , P , N ) = min ( a ( {‖ A - P ‖}_{2} - {‖ A - N ‖}_{2} ) - β {‖ P - N ‖}_{2} + γ ( \frac{1}{{R}_{p}} {\sum }_{i = 1}^{{R}_{p}} {‖ {p}_{i} - P ‖}_{2} + \frac{1}{{R}_{n}} {\sum }_{j = 1}^{{R}_{n}} {‖ {n}_{j} - N ‖}_{2} ) )$$

In addition to standard minimization between anchor and positive input (∥A−P∥₂) as well as maximization between anchor and negative input (∥A−N∥₂), the loss L is modified with k-means inspired metrics. Specifically, the inter-prior loss ∥P−N∥₂, equivalent to distance between prior centroids, can be maximized so that high- and low-risk ROIs remain distinct from each other. Likewise, the intra-prior loss

$$\frac{1}{{R}_{p}} {\sum }_{i = 1}^{{R}_{p}} {‖ {p}_{i} - P ‖}_{2} + \frac{1}{{R}_{n}} {\sum }_{j = 1}^{{R}_{n}} {‖ {n}_{j} - N ‖}_{2}$$

can be used to measure the mean voxel-wise distance from prior centroids for both high- and low-risk priors, where pᵢ and nⱼ are the feature vectors for individual high- and low-risk ROI voxels while Rₚ and Rₙ are the respective number of voxels in high- and low-risk ROIs. The intra-prior loss can be minimized to ensure the voxel-wise feature data of both priors are internally consistent. Hyperparameters α, β, and γ are weighting factors for standard triplet, inter-prior, and intra-prior losses respectively. The loss can be updated during each region growing iteration by recalculating centroids P and N from the updated high- and low-risk prior image data and continues until the overall loss exceeds a hyperparameter threshold E. Combined, these metrics can generate differences between IR prior image features while ensuring internal prior consistency.

The above-described systems and methods can be used to automatically identify PZ areas with high and low infiltration risk on medical image data, which can be used in two main ways. First, TripleSeq can generate a voxel-wise infiltration risk map which can be overlaid on medical images to highlight at-risk regions. Second, PZ regions that are most likely to be infiltrated (high-risk) and most likely to be non-infiltrated (low-risk) can be used to create infiltration risk ROIs. These infiltration risk ROIs are then used to train image-based AI models for infiltration and recurrence prediction.

For example, to supplement limited pathological ground truth data, MRI-based machine learning (ML) models for glioblastoma (GBM) pre-operative infiltration prediction often train on domain knowledge metrics like distance. However, such approaches often involve manual segmentation which is tedious, requires expert input, and is highly variable. To address these drawbacks, TripleSeq can automatically derive infiltration risk (IR) priors as surrogate for ground truth. TripleSeq iteratively searches the peritumoral region to identify candidate ROIs with high and low similarity to enhancing tumor (ET) image data. TripleSeq can be fully data-driven and not require specific MRI contrasts or image sequences as input. This makes TripleSeq suitable for automatic generation of IR priors from multiparametric MRI (mpMRI) data. TripleSeq was evaluated for its application in mpMRI with MR fingerprinting (MRF) radiomics for GBM infiltration prediction, as just one, non-limiting example.

An imaging biomarker for GBM infiltration should be consistently different between enhancing tumor (ET) and peritumor without infiltration. Furthermore, a monotonic trend should exist for peritumor with intermediate infiltrative potential, with high-risk regions having similar image features to ET and low-risk areas being dissimilar to ET.

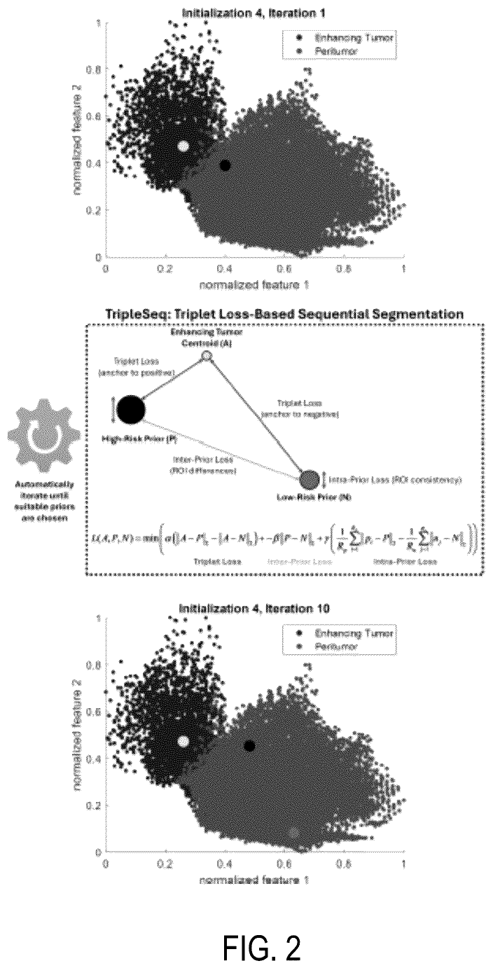

TripleSeq can employ this assumption to automatically identify IR priors. This concept, as applied to this clinical application is illustrated in FIG. 2. That is, referring to FIG. 2, voxel-wise enhancing tumor and peritumor image data can be projected into a high-dimensional image feature space, with each dimension corresponding to an image feature (e.g, T1w intensity). A modified triplet loss can be employed to identify suitable priors that represent peritumor similar to (high-risk) and dissimilar to (low-risk) enhancing tumor. In addition to the standard triplet loss, inter-prior and intra-prior losses can be added to generate image feature differences between IR priors while ensuring internal consistency within each prior.

Following whole tumor segmentation, mpMRI data can be projected voxel-wise into a high-dimensional image feature space (each dimension is a voxel-wise feature like T1w intensity) to identify the characteristic ET feature vector centroid A. The peritumor ROI (marginal area surrounding tumor core) is iteratively searched using a triplet loss, using characteristic ET feature vector A as anchor and candidate high-risk P and low-risk N feature vectors as positive and negative inputs respectively. Following selection of high- and low-risk seeds, region growing is performed with a modified triplet loss using the above equation.

$$L ( A , P , N ) = min ( a ( ‖ A - {P}_{2} ‖ - {‖ A - N ‖}_{2} ) - β {‖ P - N ‖}_{2} + γ ( \frac{1}{{R}_{p}} {\sum }_{i = 1}^{{R}_{p}} {‖ {p}_{i} - P ‖}_{2} + \frac{1}{{R}_{n}} {\sum }_{j = 1}^{{R}_{n}} {‖ {n}_{j} - N ‖}_{2} ) )$$

In addition to standard minimization between anchor (ET) and positive input (high-risk prior) and maximization between anchor and negative input (low-risk prior), the triplet loss is modified with k-means inspired metrics to include inter-prior (∥P−N∥₂; equivalent to distance between cluster centroids) and intra-prior (

$$\frac{1}{{R}_{p}} {\sum }_{i = 1}^{{R}_{p}} {‖ {p}_{i} - P ‖}_{2} - \frac{1}{{R}_{n}} {\sum }_{{j = 1}^{}}^{{R}_{p}} {‖ {n}_{j} - N ‖}_{2} ;$$

equivalent to average point-wise distance from cluster centroids) similarity terms: α, β, and γ are weighting hyperparameters for triplet, inter-prior, and intra-prior losses, respectively. The triplet loss is calculated during region growing that terminates when either inter-prior loss falls below a threshold & (priors and similar) or intra-prior loss surpasses a threshold ε₂ (ROI voxels have high variance). These metrics generate differences between IR priors while ensuring each prior is internally consistent.

### Materials and Methods

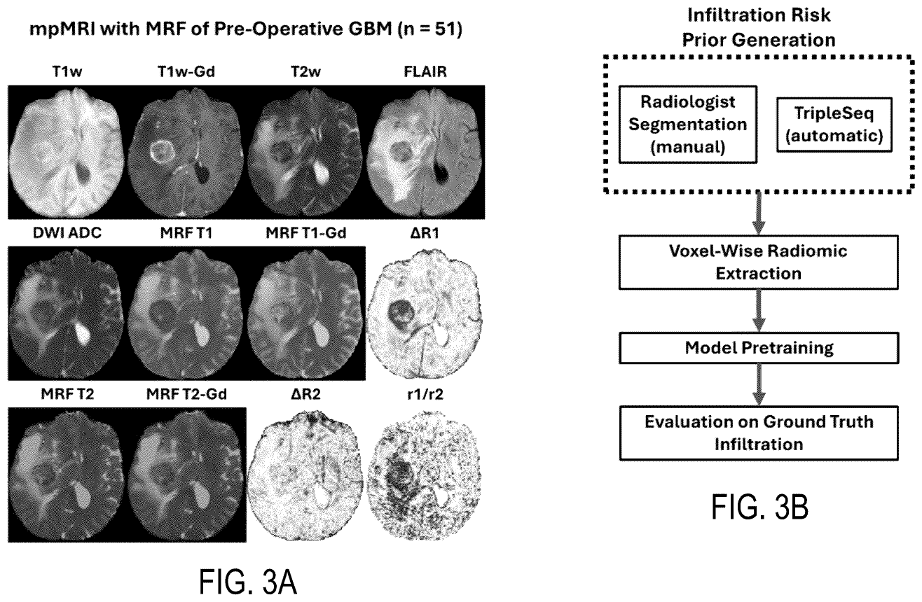

Pre-operative 3D MRF (T1 and T2; w/wo contrast) and mpMRI (T1w, T1w-Gd, T2w, FLAIR, and DWI ADC) from GBM patients (n=51) was analyzed, as illustrated in FIGS. 3A and 3B. MRI data was obtained from independent cohorts acquired between February 2017 and February 2020 (cohort 1) and between July 2022 and October 2023 (cohort 2) following IRB approval.

Following tumor segmentation into ET and peritumor, IR prior generation with TripleSeq using mpMRI, MRF, and MRF-derived delta relaxometry map, voxel-wise mpMRI radiomic features (98 per MRI sequence (n=12); 1176 total) were extracted from IR priors using a 5×5×5 sliding kernel. Features were used to train a multilayer perceptron (five FC layers) for voxel-wise classification of infiltration risk, using data from cohort 1, cohort 2, and combined cohorts.

Referring to FIG. 3A, the pre-operative MRF and mpMRI images from 51 GBM patients were analyzed; MRF-derived delta relaxometry11 maps were included in analysis. Referring to FIG. 3B, following IR prior generation via either TripleSeq or manual radiologist segmentation, voxel-wise radiomic extraction (1176 total features) was performed and a multilayer perceptron was trained to classify high- and low-risk infiltration status. Trained models were evaluated by testing on pathologically confirmed sites of non-enhancing peritumoral infiltration.

A subset (n=14) of patients had pathologically confirmed non-enhancing peritumoral infiltration identified through targeted biopsy or intra-operative 5-ALA fluorescence13: these cases were withheld from training and had ground truth infiltration ROIs (n=58) annotated by a board-certified neuroradiologist in collaboration with the operating neurosurgeon.

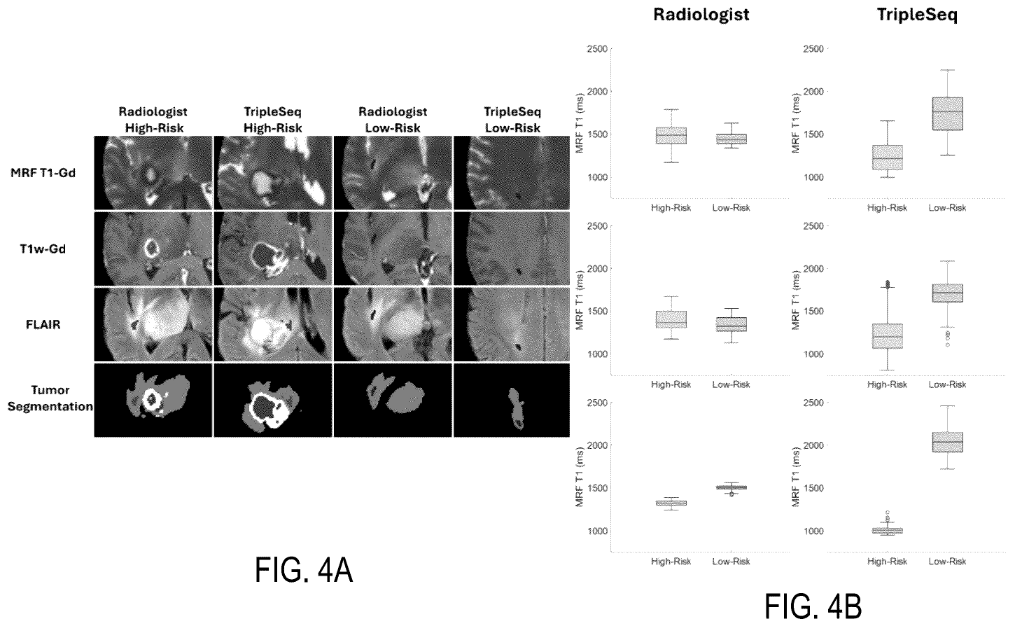

Referring to FIG. 4A, IR priors generated by manual radiologist segmentation and by TripleSeq are shown. TripleSeq selected an enhancing tumor-adjacent ROI as high-risk (red) and a distant peritumoral area with low T1w-Gd signal as low-risk (blue). Referring to FIG. 4B, a comparison of image features (MRF T1 shown) from TripleSeq and radiologist ROIs are shown. TripleSeq-generated IR priors showed consistent and clear differences between high- and low-risk priors; in comparison, manual ROI differences were ambiguous and less generalizable across patients.

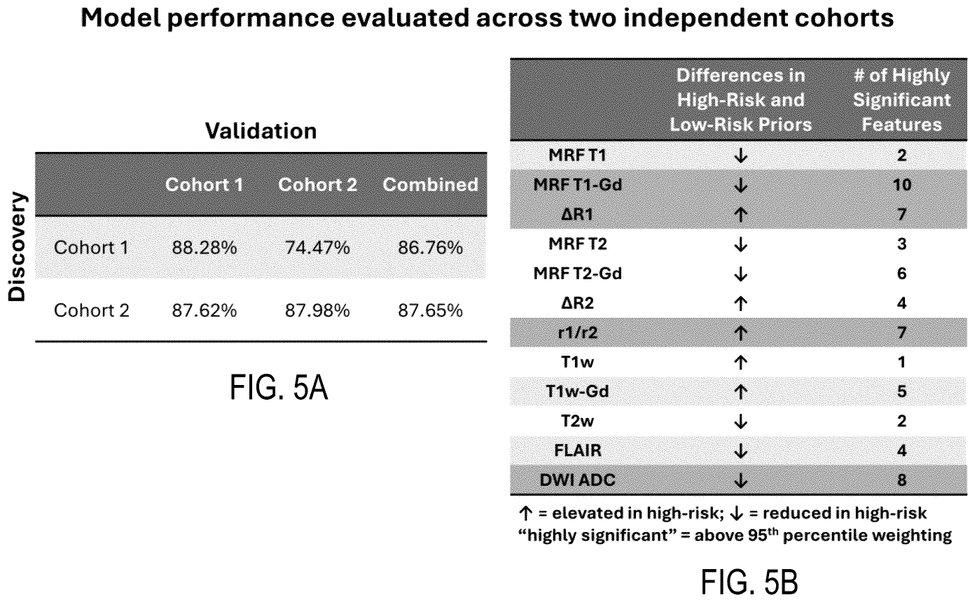

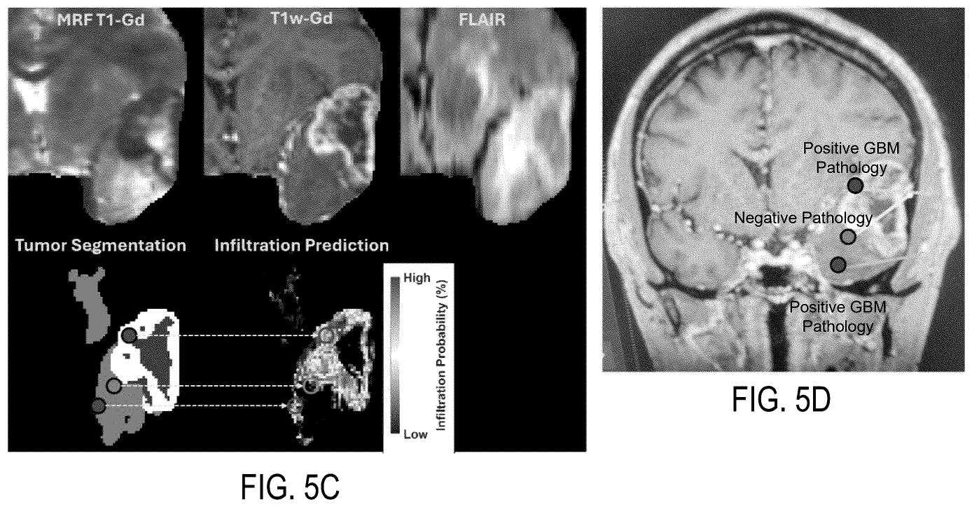

Referring to FIGS. 5A and 5B, infiltration ROIs were used to test voxel-wise classification accuracy. In particular, referring to FIG. 5A, tripleSeq-generated IR priors were used in three discovery-validation training schemes. Trained models demonstrated good test prediction (>85% mean accuracy) of ground truth infiltration status across all training schemes. Referring to FIG. 5B, characteristic mpMRI feature differences between high- and low-risk peritumor were identified (up arrow indicating greater value in high-risk, down arrow in low-risk). MRF T1-Gd, DWI ADC, r1/r2, and ΔΔR1 (highlighted) had the greatest number of highly significant features (>95th percentile weighting).

### Results

TripleSeq-generated priors showed consistent image feature trends compared to manual segmentation (FIG. 4B), with high-risk priors being similar to ET and low-risk priors being dissimilar. Mean processing time per IR prior was <1 min with TripleSeq and >5 min manually. Across discovery-validation schemes, training with TripleSeq priors led to good test prediction (>85% mean accuracy) of ground truth infiltration status (FIG. 5A). Characteristic mpMRI feature value differences between high- and low-risk peritumor (FIG. 5B) align with previously reported infiltration signatures1,2. MRF T1-Gd, ADC, r1/r2, and ΔR1 had the greatest number of highly significant features.

Finally, the model can be applied to generate whole tumor infiltration prediction maps for prospective neurosurgical or radiotherapy guidance, as illustrated in FIGS. 5C and 5D. That is, referring to FIG. 5C, the trained model generates infiltration prediction maps for prospective identification of target peritumor for neurosurgery or radiotherapy. Referring to FIG. 5D, the infiltration status of three biopsies (trajectories denoted by lines) was pathologically confirmed. All three biopsies were accurately identified on the infiltration prediction map (one positive in enhancing tumor; one positive in peritumor; one negative in peritumor).

Thus, systems and methods are provided that can provide an automatic, data-driven framework to generate infiltration risk priors for GBM infiltration prediction from mpMRI. The trained model demonstrates high prediction accuracy of ground truth infiltration and can be applied prospectively to guide neurosurgery and radiotherapy.

The above-described systems and methods present substantial advantages over existing risk stratification methods for PZ infiltration. For example, the described TripleSeq method identifies quantitative image-based differences between areas in the PZ, which are used to identify areas with high and low infiltrative risk. Because of this, the described method is particularly suitable for identifying infiltration risk metrics from quantitative image modalities including magnetic resonance fingerprinting (MRF) and CT.

The described systems and methods do not rely on explicit domain knowledge (e.g., such as distance from tumor) for PZ risk stratification. The systems and methods can be implemented to be automatic and not require manual, expert guidance (from board certified physicians). The systems and methods can be used for PZ infiltration risk stratification of any tumor type (with a defined PZ) and are not limited to specific organ systems or body regions. The described systems and method can be used with any medical image modality or input (e.g., MRI, CT, PET, etc.). For imaging methods that provide distinct image contrasts (e.g., T1- and T2-weighted MRI images), the described method can be used regardless of specific contrast input.

The described technology has been applied in an ongoing research study evaluating its effectiveness for PZ infiltration prediction in pre-operative GBM. A lab presentation detailing the described technology's initial conception and implementation is provided, as well as a conference abstract (accepted for oral presentation) detailing the initial study results.

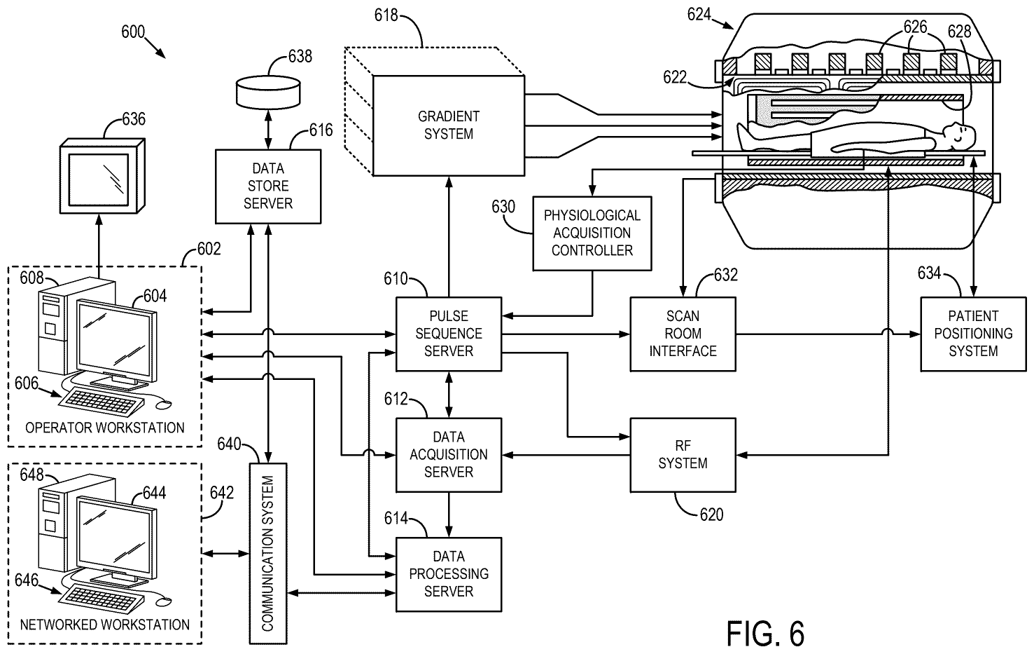

The above-described systems and methods may be used with any of a variety of imaging systems or data from such imaging systems. As one non-limiting example, the imaging system may include MR systems, and/or computer systems. Referring particularly now to FIG. 6, an example of an MRI system 600 that can implement the methods described herein is illustrated. The MRI system 600 includes an operator workstation 602 that may include a display 604, one or more input devices 606 (e.g., a keyboard, a mouse), and a processor 608. The processor 608 may include a commercially available programmable machine running a commercially available operating system. The operator workstation 602 provides an operator interface that facilitates entering scan parameters into the MRI system 600. The operator workstation 602 may be coupled to different servers, including, for example, a pulse sequence server 610, a data acquisition server 612, a data processing server 614, and a data store server 616. The operator workstation 602 and the servers 610, 612, 614, and 616 may be connected via a communication system 640, which may include wired or wireless network connections.

The MRI system 600 also includes a magnet assembly 624 that includes a polarizing magnet 626, which may be a low-field magnet. The MRI system 600 may optionally include a whole-body RF coil 628 and a gradient system 618 that controls a gradient coil assembly 622.

The pulse sequence server 610 functions in response to instructions provided by the operator workstation 602 to operate a gradient system 618 and a radiofrequency (“RF”) system 620. Gradient waveforms for performing a prescribed scan are produced and applied to the gradient system 618, which then excited gradient coils in an assembly 622 to produce the magnetic field gradients (e.g., Gₓ, G_{y}, and G_{z}) that can be used for spatially encoding magnetic resonance signals. The gradient coil assembly 622 forms part of a magnet assembly 624 that includes a polarizing magnet 626 and a whole-body RF coil 628.

RF waveforms are applied by the RF system 620 to the RF coil 628, or a separate local coil to perform the prescribed magnetic resonance pulse sequence. Responsive magnetic resonance signals detected by the RF coil 628, or a separate local coil, are received by the RF system 620. The responsive magnetic resonance signals may be amplified, demodulated, filtered, and digitized under direction of commands produced by the pulse sequence server 610. The RF system 620 includes an RF transmitter for producing a wide variety of RF pulses used in MRI pulse sequences. The RF transmitter is responsive to the prescribed scan and direction from the pulse sequence server 610 to produce RF pulses of the desired frequency, phase, and pulse amplitude waveform. The generated RF pulses may be applied to the whole-body RF coil 628 or to one or more local coils or coil arrays.

The RF system 620 also includes one or more RF receiver channels. An RF receiver channel includes an RF preamplifier that amplifies the magnetic resonance signal received by the coil 628 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 610 may receive patient data from a physiological acquisition controller 630. By way of example, the physiological acquisition controller 630 may receive signals from a number of different sensors connected to the patient, including electrocardiograph (“ECG”) signals from electrodes, or respiratory signals from a respiratory bellows or other respiratory monitoring devices. These signals may be used by the pulse sequence server 610 to synchronize, or “gate,” the performance of the scan with the subject's heartbeat or respiration.

The pulse sequence server 610 may also connect to a scan room interface circuit 632 that receives signals from various sensors associated with the condition of the patient and the magnet system. Through the scan room interface circuit 632, a patient positioning system 634 can receive commands to move the patient to desired positions during the scan.

The digitized magnetic resonance signal samples produced by the RF system 620 are received by the data acquisition server 612. The data acquisition server 612 operates in response to instructions downloaded from the operator workstation 602 to receive the real-time magnetic resonance data and provide buffer storage, so that data are not lost by data overrun. In some scans, the data acquisition server 612 passes the acquired magnetic resonance data to the data processor server 614. In scans that require information derived from acquired magnetic resonance data to control the further performance of the scan, the data acquisition server 612 may be programmed to produce such information and convey it to the pulse sequence server 610. For example, during pre-scans, magnetic resonance data may be acquired and used to calibrate the pulse sequence performed by the pulse sequence server 610. As another example, navigator signals may be acquired and used to adjust the operating parameters of the RF system 620 or the gradient system 618, or to control the view order in which k-space is sampled. In still another example, the data acquisition server 612 may also process magnetic resonance signals used to detect the arrival of a contrast agent in a magnetic resonance angiography (“MRA”) scan. For example, the data acquisition server 612 may acquire magnetic resonance data and processes it in real-time to produce information that is used to control the scan.

The data processing server 614 receives magnetic resonance data from the data acquisition server 612 and processes the magnetic resonance data in accordance with instructions provided by the operator workstation 602. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 614 are conveyed back to the operator workstation 602 for storage. Real-time images may be stored in a data base memory cache, from which they may be output to operator display 602 or a display 636. Batch mode images or selected real time images may be stored in a host database on disc storage 638. When such images have been reconstructed and transferred to storage, the data processing server 614 may notify the data store server 616 on the operator workstation 602. The operator workstation 602 may be used by an operator to archive the images, produce films, or send the images via a network to other facilities.

The MRI system 600 may also include one or more networked workstations 642. For example, a networked workstation 642 may include a display 644, one or more input devices 646 (e.g., a keyboard, a mouse), and a processor 648. The networked workstation 642 may be located within the same facility as the operator workstation 602, or in a different facility, such as a different healthcare institution or clinic.

The networked workstation 642 may gain remote access to the data processing server 614 or data store server 616 via the communication system 640. Accordingly, multiple networked workstations 642 may have access to the data processing server 614 and the data store server 616. In this manner, magnetic resonance data, reconstructed images, or other data may be exchanged between the data processing server 614 or the data store server 616 and the networked workstations 642, such that the data or images may be remotely processed by a networked workstation 642.

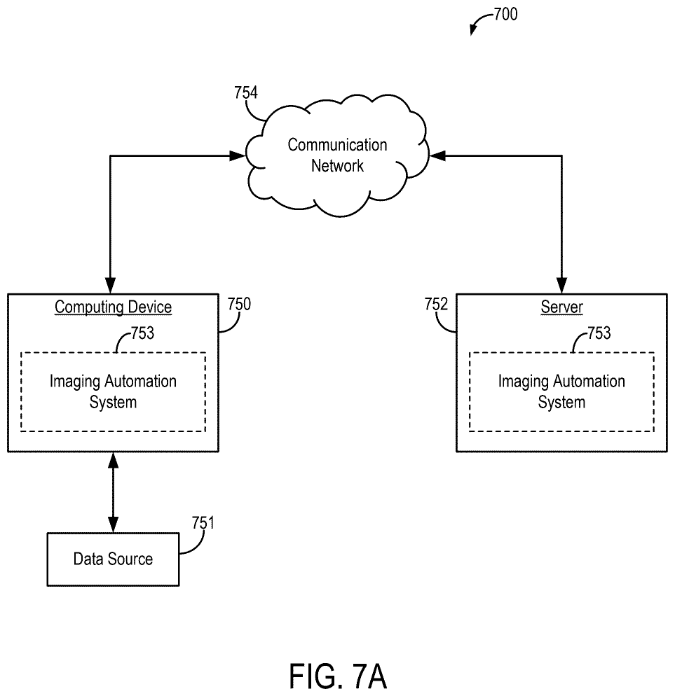

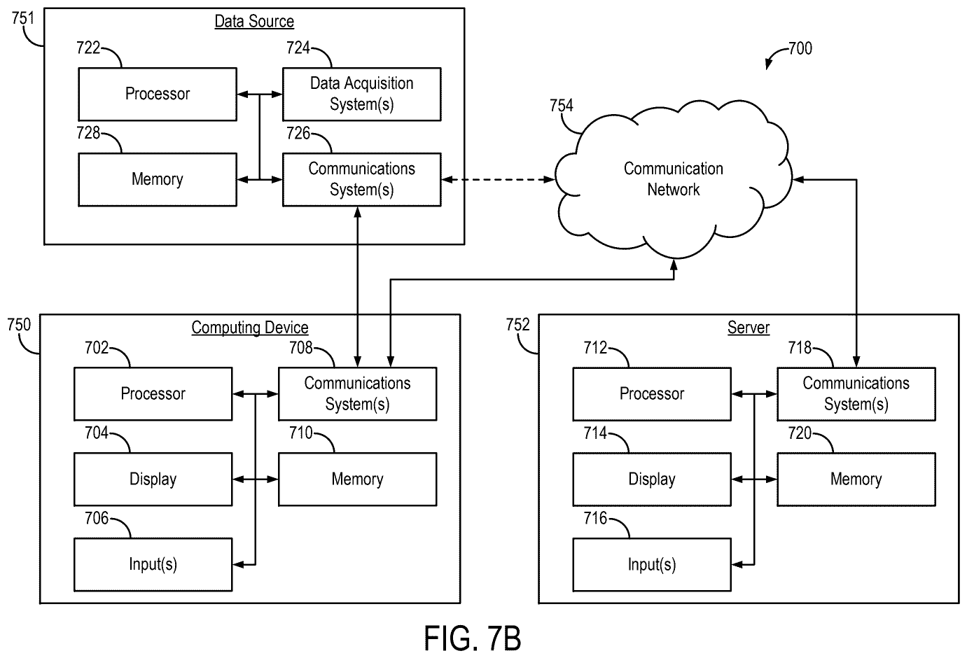

Referring now to FIG. 7A, an example of a system 700 is shown, which may be used in accordance with some aspects of the systems and methods described in the present disclosure. As shown in FIG. 7A, a computing device 750 can receive one or more types of data (e.g., signal evolution data, k-space data, receiver coil sensitivity data) from data source 751 In some configurations, computing device 750 can execute at least a portion of an imaging automation system 753 to reconstruct images from magnetic resonance data (e.g., k-space data) acquired, for example, using an LLM-generated imaging protocol. In some configurations, the imaging automation system 753 can implement an automated pipeline to provide MRI images, quantitative parameter maps, MRF maps, MRF synthetic images, image analysis reports, imaging protocols, reconstruction pipelines, etc.

Additionally or alternatively, in some configurations, the computing device 750 can communicate information about data received from the data source 751 to a server 752 over a communication network 754, which can execute at least a portion of the imaging automation system 753. In such configurations, the server 752 can return information to the computing device 750 (and/or any other suitable computing device) indicative of an output of the imaging automation system 753.

In some configurations, computing device 750 and/or server 752 can be any suitable computing device or combination of devices, such as a desktop computer, a laptop computer, a smartphone, a tablet computer, a wearable computer, a server computer, a virtual machine being executed by a physical computing device, and so on. The computing device 750 and/or server 752 can also reconstruct images from the data.

In some configurations, data source 751 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 751 can be local to computing device 750. For example, data source 751 can be incorporated with computing device 750 (e.g., computing device 750 can be configured as part of a device for measuring, recording, estimating, acquiring, or otherwise collecting or storing data). As another example, data source 751 can be connected to computing device 750 by a cable, a direct wireless link, and so on. Additionally or alternatively, in some configurations, data source 751 can be located locally and/or remotely from computing device 750, and can communicate data to computing device 750 (and/or server 752) via a communication network (e.g., communication network 754).

In some configurations, communication network 754 can be any suitable communication network or combination of communication networks. For example, communication network 754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 554 can be a local area network, a wide area network, a public network (e.g., the Internet), a private or semi-private network (e.g., a corporate or university intranet), any other suitable type of network, or any suitable combination of networks. Communications links shown in FIG. 7A can each be any suitable communications link or combination of communications links, such as wired links, fiber optic links, Wi-Fi links, Bluetooth links, cellular links, and so on.

Referring now to FIG. 7B, an example of hardware 700 that can be used to implement data source 751, computing device 750, and server 752 in accordance with some configurations of the systems and methods described in the present disclosure is shown.

As shown in FIG. 7B, in some configurations, computing device 750 can include a processor 702, a display 704, one or more inputs 706, one or more communication systems 708, and/or memory 710. In some configurations, processor 702 can be any suitable hardware processor or combination of processors, such as a central processing unit (“CPU”), a graphics processing unit (“GPU”), and so on. In some configurations, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 706 can include any suitable input devices and/or sensors that can be used to receive user input, such as a keyboard, a mouse, a touchscreen, a microphone, and so on.

In some configurations, communications systems 708 can include any suitable hardware, firmware, and/or software for communicating information over communication network 754 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some configurations, memory 710 can include any suitable storage device or devices that can be used to store instructions, values, data, or the like, that can be used, for example, by processor 702 to present content using display 704, to communicate with server 752 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 710 can have encoded thereon, or otherwise stored therein, a computer program for controlling operation of computing device 750. In such configurations, processor 702 can execute at least a portion of the computer program to present content (e.g., images, user interfaces, graphics, tables), receive content from server 752, transmit information to server 752, and so on. For example, the processor 702 and the memory 710 can be configured to perform the methods described herein.

In some configurations, server 752 can include a processor 712, a display 714, one or more inputs 716, one or more communications systems 718, and/or memory 720. In some configurations, processor 712 can be any suitable hardware processor or combination of processors, such as a CPU, a GPU, and so on. In some configurations, display 714 can include any suitable display devices, such as an LCD screen, LED display, OLED display, electrophoretic display, a computer monitor, a touchscreen, a television, and so on. In some configurations, inputs 716 can include any suitable input devices and/or sensors that can be used to receive user input, such as a keyboard, a mouse, a touchscreen, a microphone, and so on.

In some configurations, communications systems 718 can include any suitable hardware, firmware, and/or software for communicating information over communication network 754 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some configurations, memory 720 can include any suitable storage device or devices that can be used to store instructions, values, data, or the like, that can be used, for example, by processor 712 to present content using display 714, to communicate with one or more computing devices 750, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 720 can have encoded thereon a server program for controlling operation of server 752. In such configurations, processor 712 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some configurations, the server 752 is configured to perform the methods described in the present disclosure. For example, the processor 712 and memory 720 can be configured to perform the methods described herein.

In some configurations, data source 751 can include a processor 722, one or more data acquisition systems 724, one or more communications systems 726, and/or memory 728. In some configurations, processor 722 can be any suitable hardware processor or combination of processors, such as a CPU, a GPU, and so on. In some configurations, the one or more data acquisition systems 724 are generally configured to acquire data, images, or both, and can include an MRI system. Additionally or alternatively, in some configurations, the one or more data acquisition systems 724 can include any suitable hardware, firmware, and/or software for coupling to and/or controlling operations of an MRI system. In some configurations, one or more portions of the data acquisition system(s) 724 can be removable and/or replaceable.

Note that, although not shown, data source 751 can include any suitable inputs and/or outputs. For example, data source 751 can include input devices and/or sensors that can be used to receive user input, such as a keyboard, a mouse, a touchscreen, a microphone, a trackpad, a trackball, and so on. As another example, data source 751 can include any suitable display devices, such as an LCD screen, an LED display, an OLED display, an electrophoretic display, a computer monitor, a touchscreen, a television, etc., one or more speakers, and so on.

In some configurations, communications systems 726 can include any suitable hardware, firmware, and/or software for communicating information to computing device 750 (and, in some configurations, over communication network 754 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 750; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 728 can have encoded thereon, or otherwise stored therein, a program for controlling operation of medical image data source 751. In such configurations, processor 722 can execute at least a portion of the program to generate images, transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

It is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for automatic peritumoral infiltration risk stratification, comprising:
obtaining medical image data of a tumor;
segmenting the medical image data into a tumor core region and a peritumoral zone region;
projecting voxel data from the tumor core region and the peritumoral zone region into a feature space;
identifying a characteristic image data of the tumor core region as a centroid of the tumor core region voxels within the feature space;
comparing voxel feature data of the peritumoral zone region to the tumor core region centroid to rank voxels of the peritumoral zone region based on similarity to the tumor core region data;
using a modified triplet loss function to grow two distinct regions of interest within the peritumoral zone region; and
generating a report of peritumoral infiltration risk stratification using results of the modified triplet loss function to grow two distinct regions of interest within the peritumoral zone region.

2. The method of claim 1, further comprising segmenting a high-risk region by region growing in the peritumoral zone region from areas with high tumor core region similarity.

3. The method of claim 2, further comprising generating a low-risk using voxels in the peritumoral zone region with a low tumor core region similarity.

4. The method of claim 1, wherein the medical image data comprises magnetic resonance imaging (MRI) data.

5. The method of claim 1, wherein the modified triplet loss function includes an inter-prior loss term and an intra-prior loss term.

6. The method of claim 5, wherein the inter-prior loss term maximizes distance between centroids of the high-risk region and the low-risk region in the feature space.

7. The method of claim 5, wherein the intra-prior loss term minimizes average point-wise distance from centroids within each of the high-risk region and the low-risk region.

8. The method of claim 1, further comprising generating a voxel-wise infiltration risk map based on the high-risk region and the low-risk region.

9. A system for automatic peritumoral infiltration risk stratification, comprising:
a processor; and
a memory storing instructions that, when executed by the processor, cause the system to:
obtain medical image data of a tumor;
segment the medical image data into a tumor core region and a peritumoral zone region;
project voxel data from the tumor core region and the peritumoral zone region into a feature space;
identify a characteristic image data of the tumor core region as a centroid of the tumor core region voxels within the feature space;
compare each peritumoral zone region voxel's feature data to the tumor core region centroid to rank the peritumoral zone region voxels based on their similarity to the tumor core region data;
use a modified triplet loss function to grow two distinct regions of interest within the peritumoral zone region; and
generate a report using the two distinct regions of interest within the peritumoral zone region, wherein the report includes a high-risk region segmented by region growing peritumoral zone region areas with high tumor core region similarity, and a low-risk region generated using peritumoral zone region voxels with low tumor core region similarity.

10. The system of claim 9, wherein the medical image data comprises magnetic resonance imaging (MRI) data including at least one of T1-weighted, T2-weighted, fluid-attenuated inversion recovery (FLAIR), diffusion-weighted imaging (DWI), or magnetic resonance fingerprinting (MRF) sequences.

11. The system of claim 9, wherein the modified triplet loss function includes an inter-prior loss term and an intra-prior loss term.

12. The system of claim 11, wherein the inter-prior loss term maximizes distance between centroids of the high-risk region and the low-risk region in the feature space.

13. The system of claim 11, wherein the intra-prior loss term minimizes average point-wise distance from centroids within each of the high-risk region and the low-risk region.

14. The system of claim 9, wherein the instructions, when executed by the processor, further cause the system to generate a voxel-wise infiltration risk map based on the high-risk region and the low-risk region.

15. The system of claim 14, wherein the instructions, when executed by the processor, further cause the system to display the voxel-wise infiltration risk map overlaid on an anatomical image of the tumor.

16. A non-transitory computer-readable medium storing instructions that, when executed by a processor, cause the processor to perform a method for automatic peritumoral infiltration risk stratification, the method comprising:
obtaining medical image data of a tumor;
segmenting the medical image data into a tumor core region and a peritumoral zone region;
projecting voxel data from the tumor core region and the peritumoral zone region into a feature space;
identifying a characteristic image data of the tumor core region as a centroid of the tumor core region voxels within the feature space;
comparing each peritumoral zone region voxel's feature data to the tumor core region centroid to rank the peritumoral zone region voxels based on their similarity to the tumor core region data; and
using a modified triplet loss function to grow two distinct regions of interest within the peritumoral zone region, wherein the two distinct regions include at least a high-risk region segmented by region growing peritumoral zone region areas with high tumor core region similarity, and a low-risk region generated using peritumoral zone region voxels with low tumor core region similarity.

17. The non-transitory computer-readable medium of claim 16, wherein the modified triplet loss function includes an inter-prior loss term and an intra-prior loss term.

18. The non-transitory computer-readable medium of claim 17, wherein the inter-prior loss term maximizes distance between centroids of the high-risk region and the low-risk region in the feature space.

19. The non-transitory computer-readable medium of claim 17, wherein the intra-prior loss term minimizes average point-wise distance from centroids within each of the high-risk region and the low-risk region.

20. The non-transitory computer-readable medium of claim 16, wherein the method further comprises generating a voxel-wise infiltration risk map based on the high-risk region and the low-risk region, and displaying the voxel-wise infiltration risk map overlaid on an anatomical image of the tumor.
